(19) United States
(12) Patent Application Publication
(10) Pub. No.: US 2020/0023184 A1
(43) Pub. Date: Jan. 23, 2020

(54) METHOD OF FORMING AN ARTERIOVENOUS CONNECTION

(71) Applicant: NOVINTUM MEDICAL TECHNOLOGY GMBH, Schaffhausen (CH)

(72) Inventor: Nicholas INSTON, Birmingham West Midlands (GB)

(21) Appl. No.: 16/498,123

(22) PCT Filed: Apr. 6, 2017
(86) PCT No.: PCT/EP2017/058302

§ 371 Date: Sep. 26, 2019

(51) Int. Cl. A61N 1/36; A61N 1/04

(52) U.S. Cl. CPC A61N 1/36031; A61N 1/0472; A61N 1/36034; A61N 1/0456

(57) ABSTRACT

A method of forming an arteriovenous connection in a subject, comprising electrically stimulating vessels including at least one artery of the subject and at least one vein of the subject, and subsequently fluidly connecting the at least one artery to the at least one vein.

This invention relates to a method of forming an arteriovenous connection in a subject, where such a connection may be made prior to hemodialysis, for example.

### BACKGROUND

A vascular access is required for hemodialysis treatments. In particular, a vascular access permits the removal and the return of blood during hemodialysis, where the blood is passed through a filter (i.e. a dialyzer) after removal of the blood from the subject and prior to return of the blood to the subject. A vascular access permits a continuous flow of a large volume of blood to circulate through the filter (typically around 1 pint of blood per minute).

A vascular access may be a simple venous catheter that is inserted into a vein of the subject. Such arrangements are typically only employed for short-term use. Other types of vascular access may be surgically created, and include arteriovenous connections between an artery and a vein of the subject. An arteriovenous connection is advantageous in that the pressure provided by the artery increases the pressure and volume of blood flowing through the vein. Over a maturation period following creation of the arteriovenous connection, the vein grows larger and stronger, making it more suitable for regular hemodialysis. For example, a mature vein (of an arteriovenous connection) may be larger than regular veins, thereby permitting easier, more reliable access with a cannula. Additionally, a mature vein may be less susceptible to collapse when subjected to regular piercing by a cannula, as would be necessary for regular hemodialysis treatments.

One type of arteriovenous connection is an arteriovenous fistula (AVF) where a direct connection is surgically created between a vein and an artery of the subject. An AVF is a particularly advantageous arteriovenous connection as it provides good blood flow for dialysis, typically lasts longer than other types of vascular access and is less likely to get infected or cause blood clots compared with other types of vascular access. Once mature, a pair of cannulas may be inserted into the vein so that hemodialysis (or other procedures) may be performed. The required maturation of an AVF may take several months, and certain AVFs may fail to mature to the required degree.

Another type of arteriovenous connection is an arteriovenous graft (AV graft). An AV graft may be employed if an AVF fails to mature in a patient. An AV graft is a surgically-created connection between a vein and an artery via an intermediate conduit, such as a plastic tube. An AV graft may mature to a satisfactory degree over several weeks and, once matured, may last several years.

Rates of failure to mature (FTM) associated with arteriovenous connections are high. For example, up to 50% of AVFs created may never be suitable for dialysis.

It is an object of certain embodiments of the present invention to overcome certain disadvantages associated with the prior art.

It is an object of certain embodiments of the present invention to provide a method of forming an arteriovenous connection that has an improved (i.e. lower) FTM rate relative to prior art methods.

### BRIEF SUMMARY OF THE DISCLOSURE

In accordance with an aspect of the present invention there is provided a method of forming an arteriovenous connection in a subject, comprising:

electrically stimulating vessels including at least one artery of the subject and at least one vein of the subject; and

subsequently fluidly connecting the at least one artery to the at least one vein.

Methods according to embodiments of the present invention may improve (i.e. lower) failure to mature (FTM) rates of vessels subjected to electrical stimulation and subsequently formed into an arteriovenous connection. The electrical stimulation may be applied at a level and/or for a time and/or be of a form that is sufficient to stimulate the venous muscle pump of the subject.

The step of fluidly connecting the at least one artery to the at least one vein may comprise directly connecting the at least one artery to the at least one vein to form an arteriovenous fistula (AVF). In certain embodiments, the AVF may be a brachiocephalic fistula (i.e. above the elbow of the subject). Alternatively, the AVF may be a radiocephalic fistula (i.e. at the wrist of the subject).

In other embodiments, the step of fluidly connecting the at least one artery to the at least one vein may comprise connecting the at least one artery to the at least one vein with an intermediate conduit to form an arteriovenous graft.

In certain embodiments, the vessels may be electrically stimulated for at least 30 seconds, between 1 and 3 minutes, or for about 2 minutes.

The method may further comprise applying a constriction to a limb of the subject that includes the at least one artery and the at least one vein, wherein the constriction is applied after electrically stimulating the vessels and before fluidly connecting the at least one artery and the at least one vein. The constriction may be applied to the limb such that at least some of the vessels that have been electrically stimulated are disposed between the constriction and a free end of the limb. In certain embodiments, the constriction may be applied for at least 1 minute, between 2 and 4 minutes, or about 3 minutes. In certain embodiments, applying the constriction may comprise applying a tourniquet.

Electrically stimulating vessels may include generating an adjustable electrical output signal with an electrical stimulation apparatus, the signal including an adjustable output voltage, an adjustable current, and an adjustable output voltage waveform configured to elicit a physiological response in the subject.

The electrical stimulation apparatus may comprise:
(i) a powered signal generator configured to generate the adjustable electrical output signal; and
(ii) at least two electrodes in electrical communication with the signal generator and configured to be placed in electrical communication with the subject.

Electrically stimulating vessels may include:
electrically connecting the at least two electrodes to the subject; and
transmitting the output signal to the subject via the at least two electrodes to elicit the physiological response.

The method may further comprise:
monitoring biological electrical feedback from the subject in the form of the subject's biological electrical resistance and/or capacitance;
comparing the electrical feedback from the subject with the transmitted output signal;
adjusting subsequent output signals to be sent to the subject based on the comparison between the transmitted output signal and the electrical feedback; and
transmitting said subsequent output signals to the subject via the at least two electrodes.

The step of monitoring the biological electrical feedback from the subject may be performed by an electrical feedback system that is optionally in the powered signal generator.

The step of comparing the electrical feedback from the subject with the transmitted output signal may be performed using a microprocessor that is optionally integrated in the powered signal generator.

The step of electrically connecting the at least two electrodes to the subject may comprise connecting a first electrode to the hand of the subject and connecting a second electrode to the arm of the subject. Connecting the first electrode to the hand of the subject may comprise connecting the first electrode to a palmar surface of the hand of the subject.

The step of electrically stimulating vessels may comprise providing a variable output voltage to the subject between 0 and 90 volts, and optionally between 0 and 40 volts.

The step of electrically stimulating vessels may comprise providing a variable current to the subject that is less than 1 milliamp.

The subject may be a human.

In accordance with another aspect of the present invention, there is provided a method comprising:
forming an arteriovenous connection according to any method described above; and
accessing the at least one vein.

Accessing the at least one vein may comprise cannulating the at least one vein.

In embodiments where an arteriovenous graft is formed, accessing the at least one vein may comprise cannulating the intermediate conduit.

The method may comprise removing a fluid from and/or introducing a fluid to the accessed vein. Accessing the at least one vein may comprise forming a first access port and a second access port in the vein. The method may comprise removing a fluid from the at least one vein through the first access port and introducing a fluid into the at least one vein through the second access port. The removed and/or introduced fluid may be or include blood.

The method may comprise performing hemodialysis on the blood after removal of the blood from the at least one vein and before introduction of the blood into the at least one vein.

The step of accessing the at least one vein may be performed after a maturation period following the step of forming the arteriovenous connection. In certain embodiments, the maturation period may be at least 2 weeks, and preferably between 4 and 7 weeks. In other embodiments, the maturation period may be at least 1 month.

### BRIEF DESCRIPTION OF THE DRAWINGS

Embodiments of the invention are further described hereinafter with reference to the accompanying drawings, in which:

FIG. 1 is a top front isometric view of an example embodiment of an electrical stimulation apparatus according to an embodiment of the present invention;

FIG. 2 is a top isometric view of the electrical stimulation apparatus of FIG. 1, showing the cover of the electrical signal generator in an open position to expose the internal circuitry and electrical components of the example electrical signal generator;

FIG. 3 is another top isometric view of the electrical stimulation apparatus of FIG. 1;

FIG. 4 is a another top front isometric view of the electrical stimulation apparatus of FIG. 1, showing the apparatus ready for use wherein a patient has her fingertips placed in containers of electrolyte solution that are electrically connected to the signal generator of the apparatus;

FIG. 5 is a another top front isometric view of the electrical stimulation apparatus of FIG. 1, showing the apparatus in use and illustrating the distending and protruding of the patient's veins;

FIG. 6 is an electrical schematic of an embodiment of a signal generator of the electrical stimulation apparatus according to an embodiment of the present invention;

FIG. 7 is a waveform graph of the output voltage vs. time for one cycle of the output signal, such as generated by the signal generator shown in FIG. 6;

FIG. 8 is a waveform graph illustrating another example waveform;

FIG. 9 is a waveform graph illustrating another example waveform;

FIG. 10 is a top view of an example embodiment of direct electrode placement for electrical stimulation;

FIG. 11 is a top view of an example embodiment of a kit comprising the electrical stimulation apparatus according to an embodiment of the present invention;

FIG. 12 is a table illustrating example outputs from the electrical stimulation apparatus according to an embodiment of the present invention;

FIG. 13 is a waveform graph illustrating another example waveform;

FIG. 14 is an electrical schematic of another embodiment of a signal generator of the electrical stimulation apparatus according to an embodiment of the present invention;

FIG. 15 is a waveform graph illustrating another example waveform; and

FIG. 16 is a waveform graph illustrating another example waveform.

### DETAILED DESCRIPTION

In accordance with an aspect of the present invention, there is provided a method of forming an arteriovenous connection in a subject, comprising electrically stimulating vessels including at least one artery of the subject and at least one vein of the subject, and subsequently fluidly connecting the at least one artery to the at least one vein.

Methods according to embodiments of the present invention may improve (i.e. lower) failure to mature (FTM) rates of vessels subjected to electrical stimulation and subsequently formed into an arteriovenous connection. The electrical stimulation may be applied at a level and/or form sufficient to stimulate the venous muscle pump of the subject.

In a pilot study incorporating a method according to an embodiment of the present invention, electrical stimulation was applied to the vessels of a subject for 2 minutes using a first electrode on the thenar eminence and a second electrode on the mid bicep of the subject, where the first electrode and second electrode were connected to the Veinplicity® electrical stimulation device. Following electrical stimulation, a tourniquet was applied and the electrodes were removed. The tourniquet provided a constrictive force around the arm of the subject for 3 minutes. The tourniquet was subsequently removed and arteriovenous fistula (AVF) surgery was performed on the arm in accordance with standard practice. Outcomes at the end of surgery and 6 weeks following surgery were assessed, as were any adverse effects.

Table 1 below summarises the patients that were the subject of the pilot study.

TABLE 1

| Demographics | Results |
|---|---|
| Age (mean) | 60 yrs. +/-14SD |
| Gender (M:F) | 3:1 |
| Fashioning of AVF | 4 radiocephalic AVF, 9 brachiocephalic AVF |
| Past medical history: | |
| Diabetic | 58% |
| Hypertensive | 75% |
| IHD | 41.6% |
| PVD | 17% |
| CVD | 25% |
| Dialysis status: | |
| Predialysis | 8 (1 conservative treatment) |
| On Dialysis | 5 (1 died unrelated) |

Table 2 summarises the parameters used and assessed outcomes.

TABLE 2

| Outcome | Results |
|---|---|
| Veinplicity ® setting mean | 8 +/-1.4SD |
| Time of application | 2 minutes |
| Patient tolerability | |
| (0/5 no discomfort to 5/5 intolerable pain) | 1.44 +/-1.35SD |
| Clinical signs range 0-5 | |
| 0 no sensation | |
| 1 mild twitch | |
| 2 expected twitch | |
| 3 good twitch | 0.92 +/-0.64SD |
| 4 excessive twitch | |
| 5 excessive shaking | |

Table 3 shows a summary of evaluation scores.

TABLE 3

| Evaluation Score | Results |
|---|---|
| (5/5 easier to 0/5 unhappy) | |
| Vein identification | 3.65/5 |
| Artery identification | 3.7/5 |
| Surgery satisfaction | 4.1/5 |
| Technical success | 100% |
| Patency at 6 weeks | 100% |

In summary, participant median age was 60 years (ranging from 33-72, M:F ratio 3:1). Four radiocephalic and nine brachiocephalic fistulas were formed. The electrical stimulation procedure was tolerated well by all patients. It was observed that uremic patients required increased levels of electrical stimulation as compared to its use in healthy control subjects. Surgeon satisfaction was high for both ease of use (4.2/5) and vessel identification (3.6/5 for vein and 3.8/5 for artery). All AVFs had a palpable thrill at the end of surgery and at 6 weeks thereafter. Primary patency was 100% with all AVFs maturing.

Embodiments of the invention include methods performed according to the pilot study described above. In other embodiments, variations in the method may be employed. In particular, the step of fluidly connecting the at least one artery to the at least one vein may comprise connecting the at least one artery to the at least one vein with an intermediate conduit to form an arteriovenous graft.

In certain embodiments, the vessels may be electrically stimulated for any suitable period of time. The electrical stimulation may be applied at a level and/or for a time and/or be of a form that is sufficient to stimulate the venous muscle pump of the subject. In particular embodiments, the vessels may be electrically stimulated for at least 30 seconds, between 1 and 3 minutes, or for about 2 minutes.

The method may not include the application of a constriction (e.g. a tourniquet). In certain embodiments, however, the method may further comprise applying a constriction to a limb of the subject that includes the at least one artery and the at least one vein, wherein the constriction is applied after electrically stimulating the vessels and before fluidly connecting the at least one artery and the at least one vein. The constriction may be applied to the limb such that at least some of the vessels that have been electrically stimulated are disposed between the constriction and an end of the limb. In certain embodiments, the constriction may be applied for at least 1 minute, between 2 and 4 minutes, or about 3 minutes. In certain embodiments, applying the constriction may comprise applying a tourniquet.

The electrical stimulation may be applied by any suitable means. In certain embodiments, the electrical stimulation may be applied using the Veinplicity® device (www.veinplicity.com). In certain embodiments, the electrical stimulation may be applied using a device described in or in accordance with a method described in WO-A-2015/041966 (Rising Tide Foundation) or WO-A-2016/126467 (Rising Tide Foundation) the disclosures of which are hereby incorporated by reference in their entirety. In certain embodiments, the electrical stimulation may be applied using an electrical stimulation apparatus configured to provide an adjustable electrical output signal with an electrical stimulation apparatus, the signal including an adjustable output voltage, an adjustable current, and an adjustable output voltage waveform configured to elicit a physiological response in the subject. The electrical stimulation apparatus may comprise a powered signal generator configured to generate the adjustable electrical output signal, and at least two electrodes in electrical communication with the signal generator and configured to be placed in electrical communication with the subject. Electrically stimulating vessels may include electrically connecting the at least two electrodes to the subject, and transmitting the output signal to the subject via the at least two electrodes to elicit the physiological response.

Given that a particular subject has a specific physiology that will determine the penetration depth of and response to the electrical stimulation, it may be preferable to include a feedback loop based on a measured response of the subject to adjust the output signal to achieve a desired effect. In such embodiments, the method may further comprise:
monitoring biological electrical feedback from the subject in the form of the subject's biological electrical resistance and/or capacitance;
comparing the electrical feedback from the subject with the transmitted output signal;
adjusting subsequent output signals to be sent to the subject based on the comparison between the transmitted output signal and the electrical feedback; and
transmitting said subsequent output signals to the subject via the at least two electrodes.

The step of monitoring the biological electrical feedback from the subject may be performed by an electrical feedback system that is optionally in the powered signal generator. The step of comparing the electrical feedback from the subject with the transmitted output signal may be performed using a microprocessor that is optionally integrated in the powered signal generator.

The step of electrically connecting the at least two electrodes to the subject may comprise connecting a first electrode to the hand of the subject and connecting a second electrode to the arm of the subject. Connecting the first electrode to the hand of the subject may comprise connecting the first electrode to a palmar surface of the hand of the subject. Other electrode arrangements may be used in accordance with embodiments of the present invention.

The step of electrically stimulating vessels may comprise providing a variable output voltage to the subject between 0 and 90 volts, and optionally between 0 and 40 volts.

The step of electrically stimulating vessels may comprise providing a variable current to the subject that is less than 1 milliamp.

In other embodiments, the electrical stimulation may be provided by any apparatus or method described below with reference to the figures.

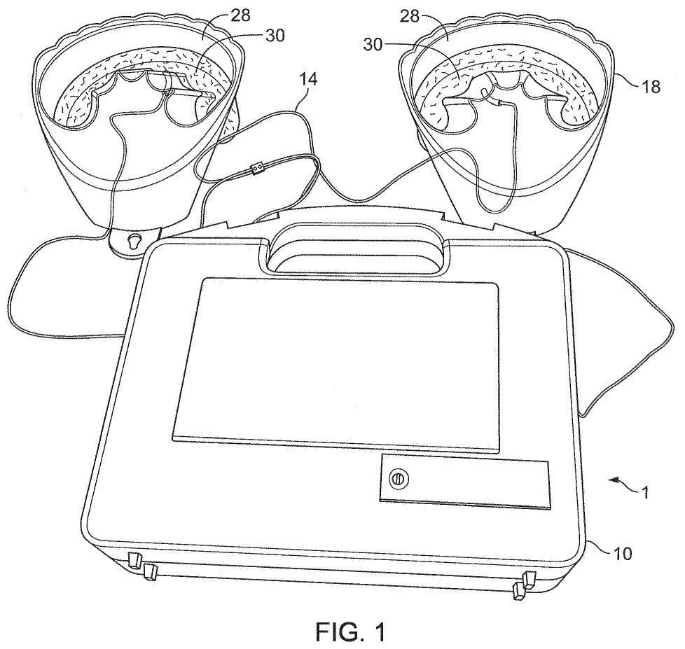

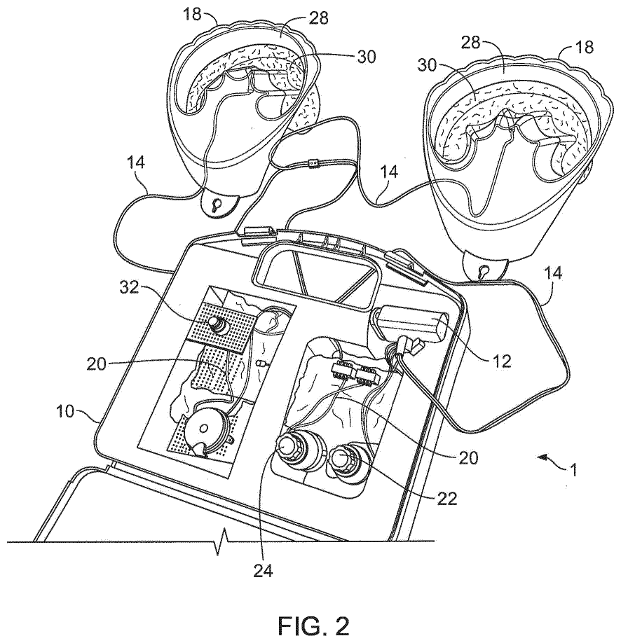

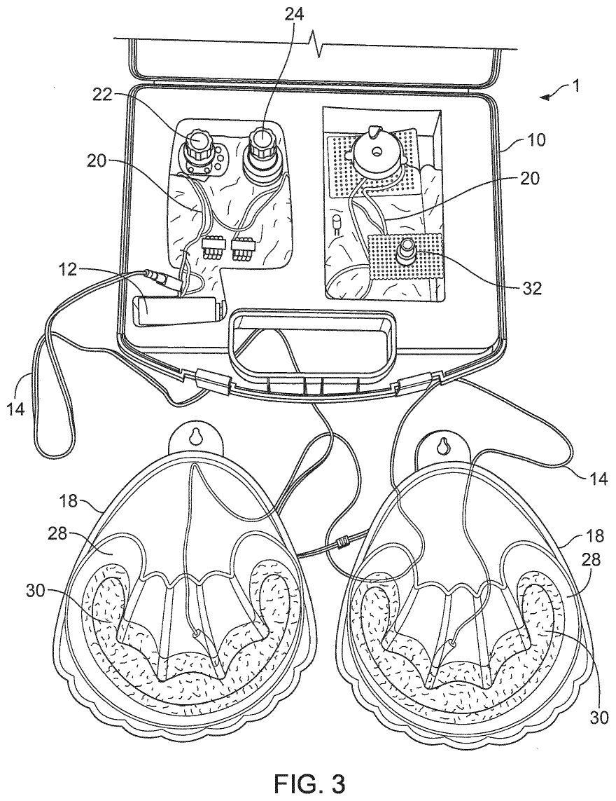

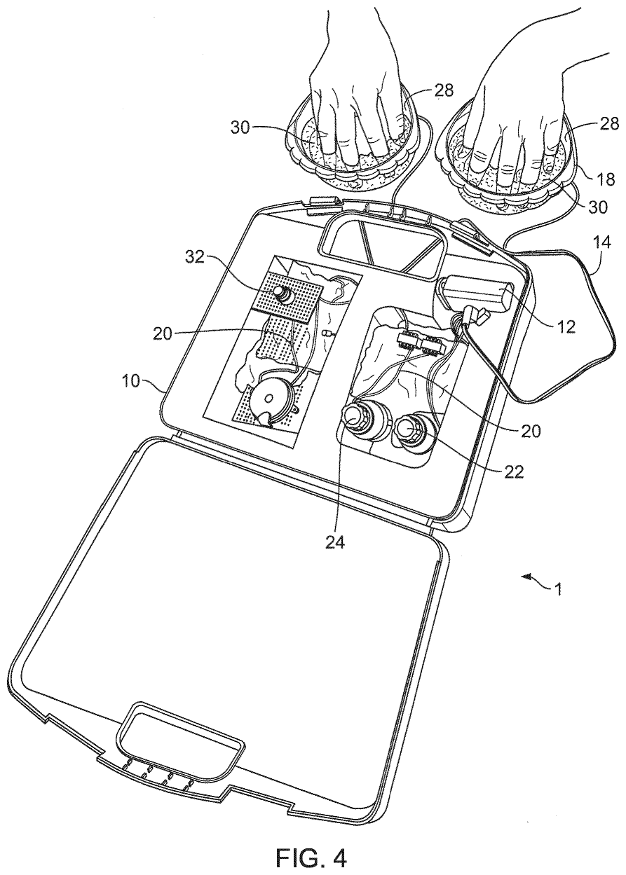

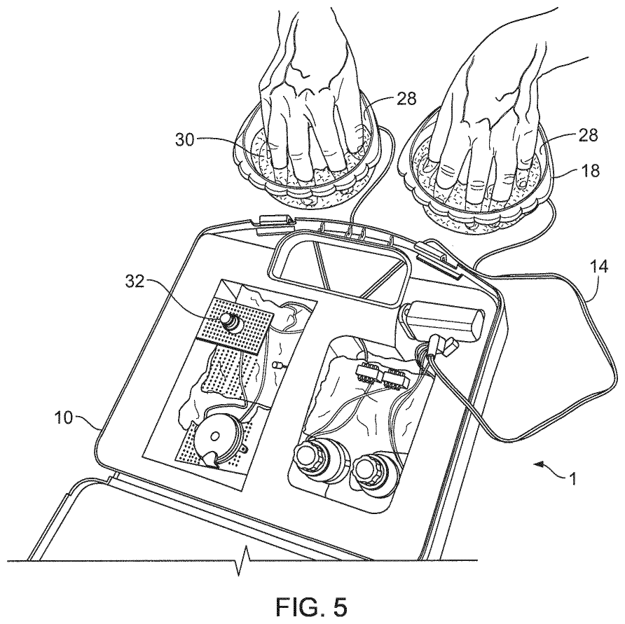

Referring to FIGS. 1-5, in general, disclosed herein is one embodiment of an electrical stimulation apparatus 1 configured to deliver an electrical signal through the arms or other limbs of a patient, e.g. from one limb, up through the limb, across the spine and down the other limb. Electrical stimulation using the electrical stimulation apparatus 1 may cause the veins in the hands, arms, legs or feet of the patient to distend or expand. In doing so, the stimulation apparatus may make the peripheral veins in the arms, hands, legs or feet of the patient more visible, thereby improving venous access. The apparatus is generally placed in electrical communication with a patient's hands and/or arms (or other limbs) by a pair of electrodes or other means that connects the device to the patient's arms or feet to deliver a predetermined electrical signal through the electrically connected limbs of the patient.

In general, the electrical stimulation apparatus 1 comprises an electrical signal generator 10, a power supply 12 in electrical communication with the signal generator and configured to supply power thereto, at least a pair of electrical leads 14 connected at a proximal end to a plurality of electrical output terminals 16 of the electrical signal generator, and at least a pair of electrodes 18 connected to a distal end of each of the electrical leads 14.

The electrical power supply 12 may be a portable power supply, such as for example a 9-volt battery, other voltage battery, or rechargeable battery. Alternatively, the power supply may utilize a standard electrical power cord that plugs into a typical power outlet in a wall.

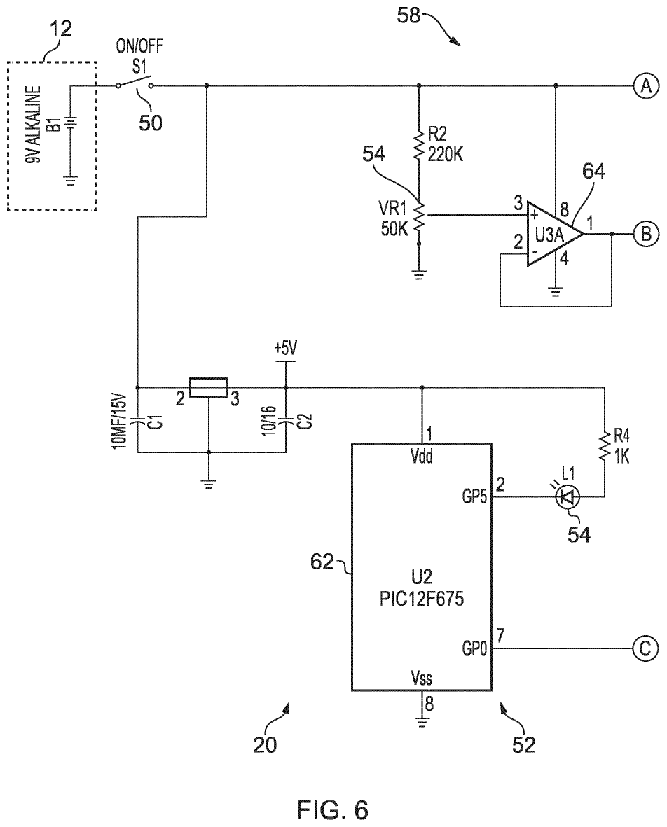

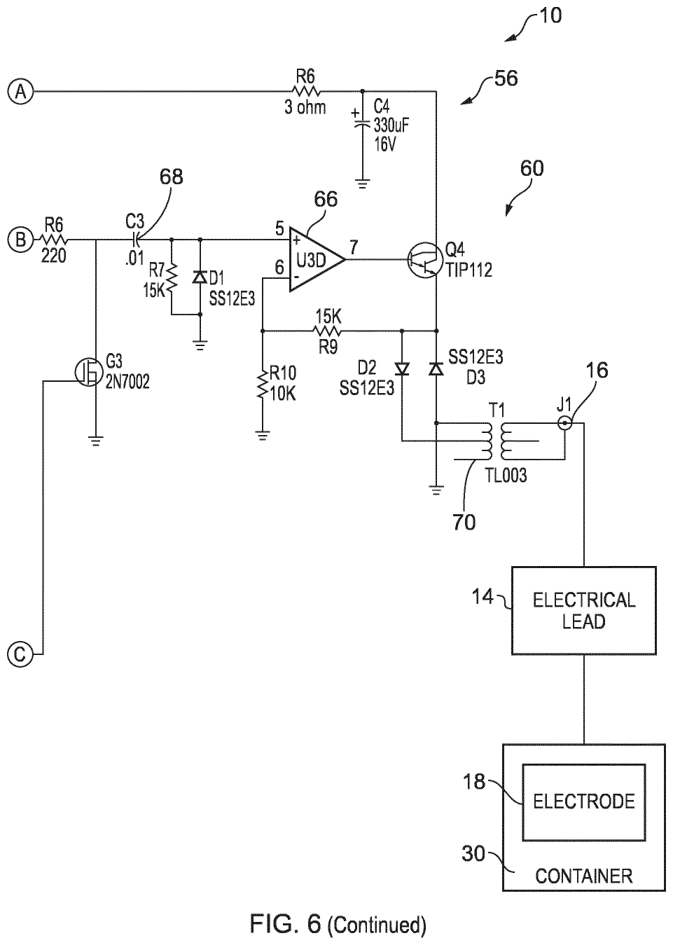

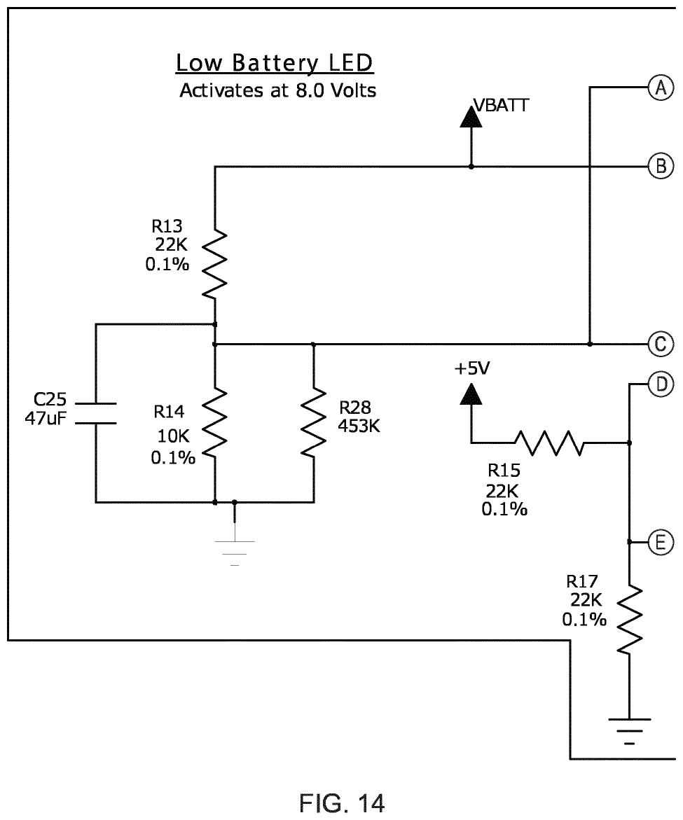

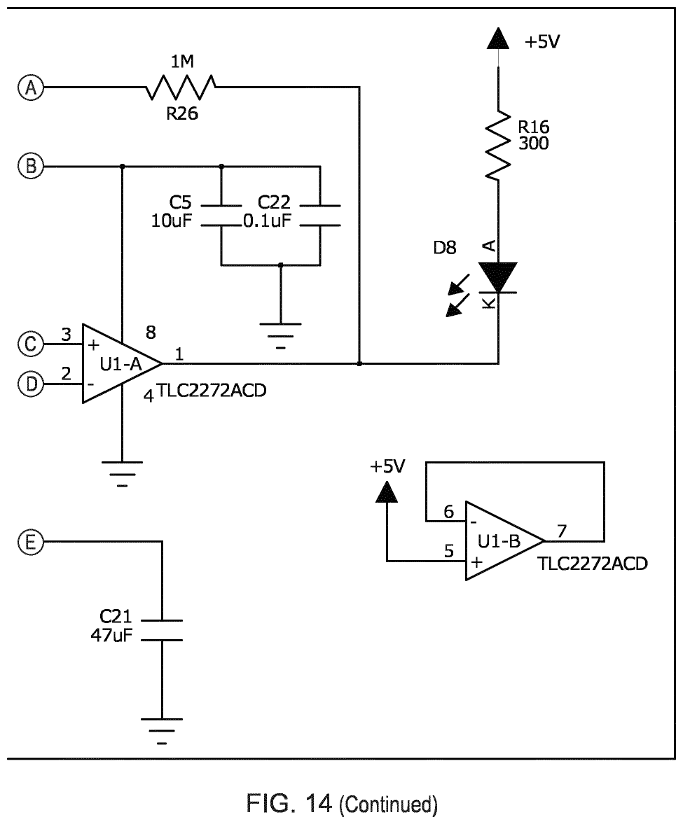

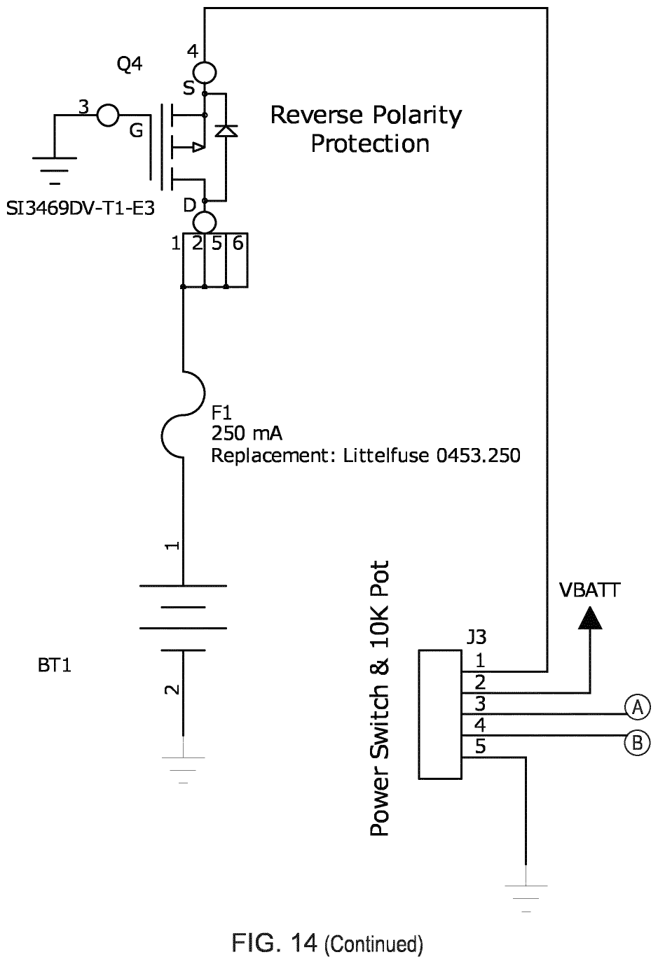

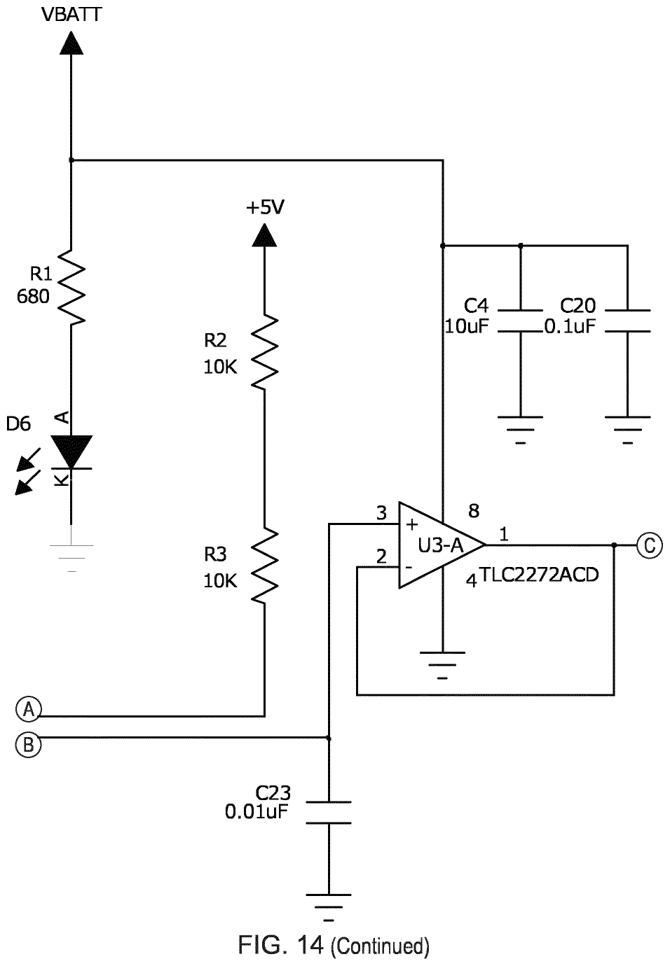

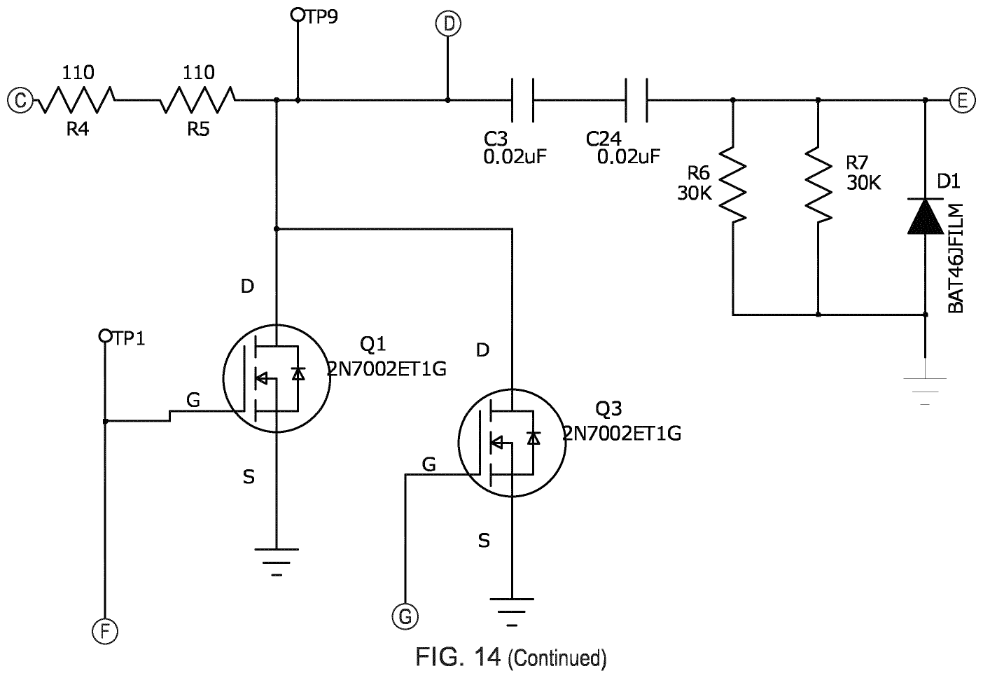

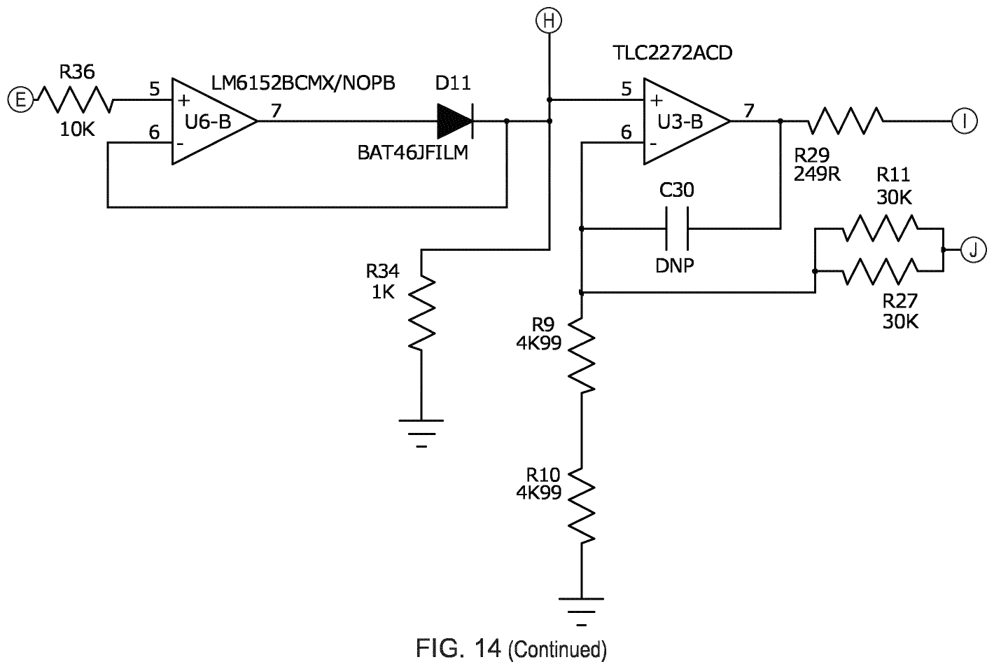

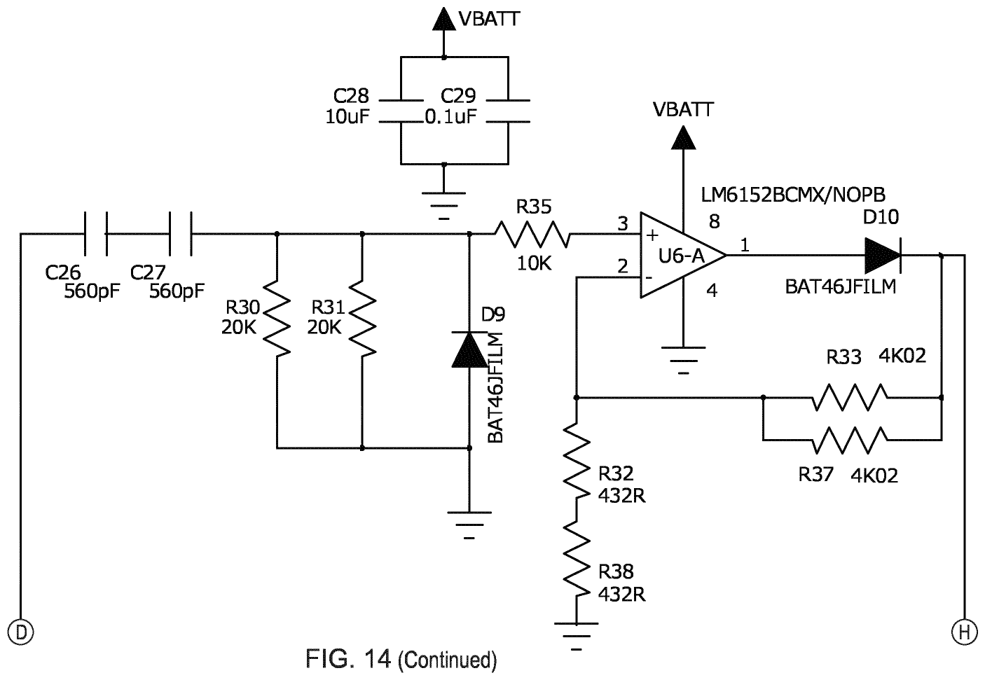

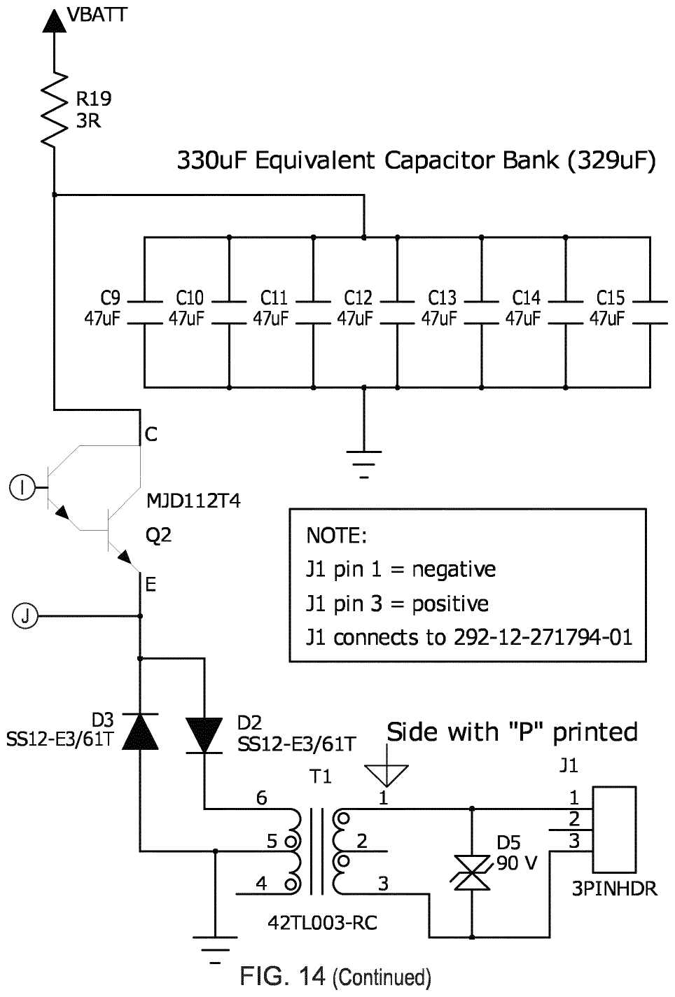

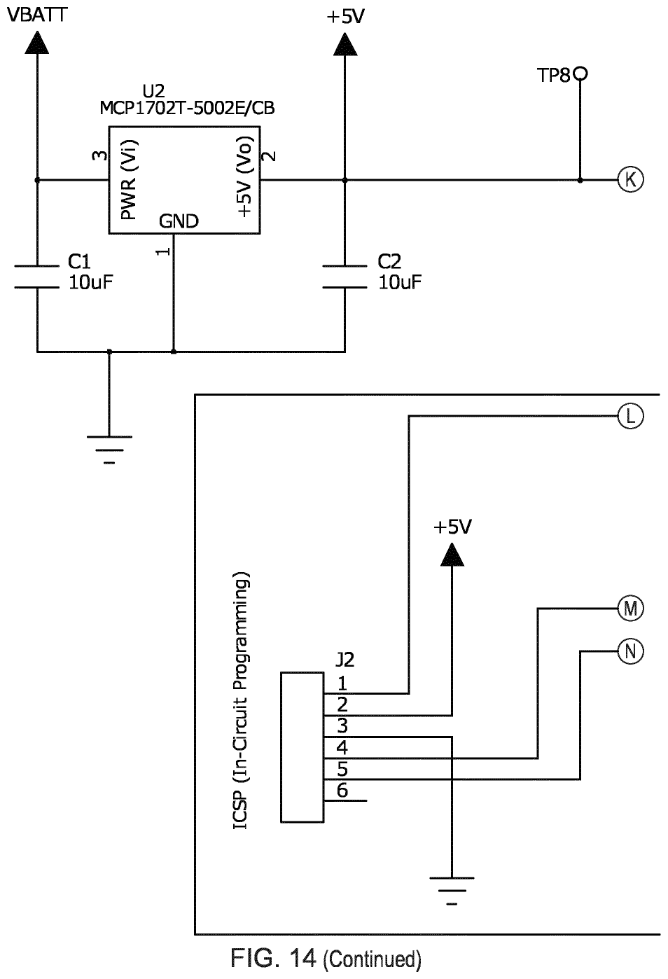

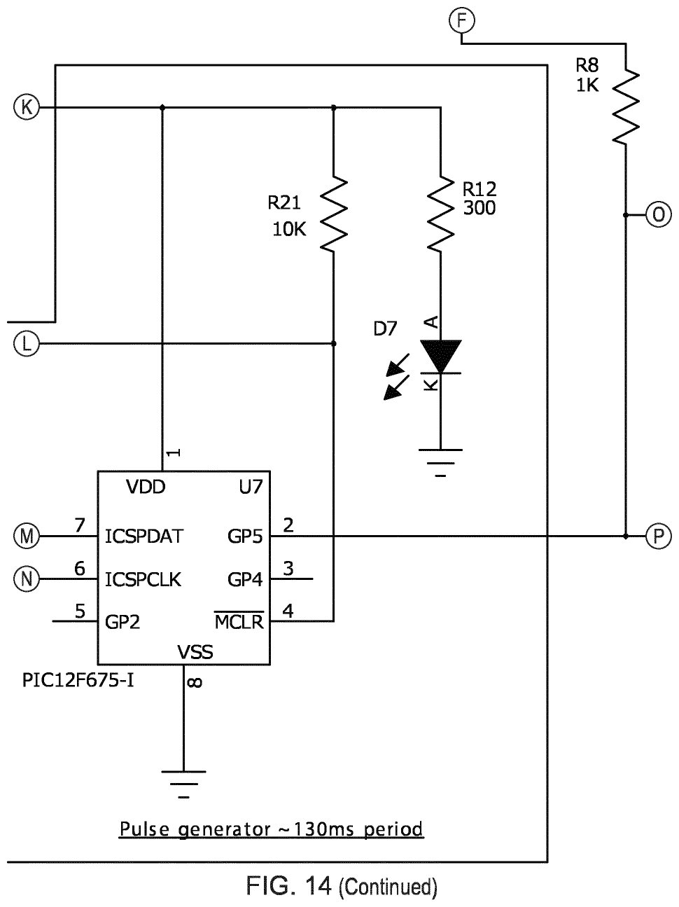

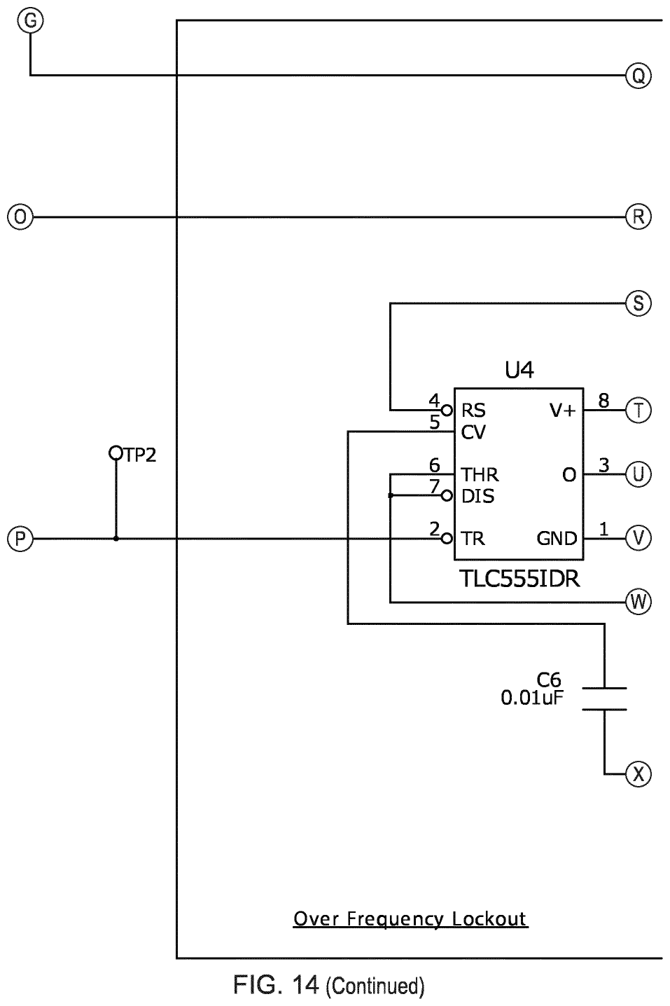

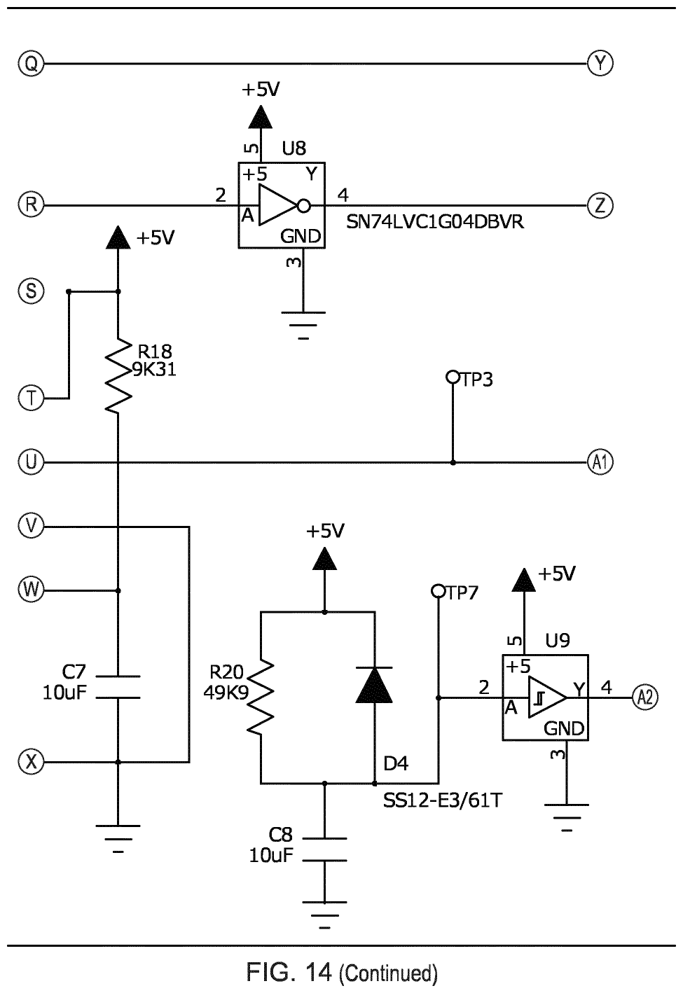

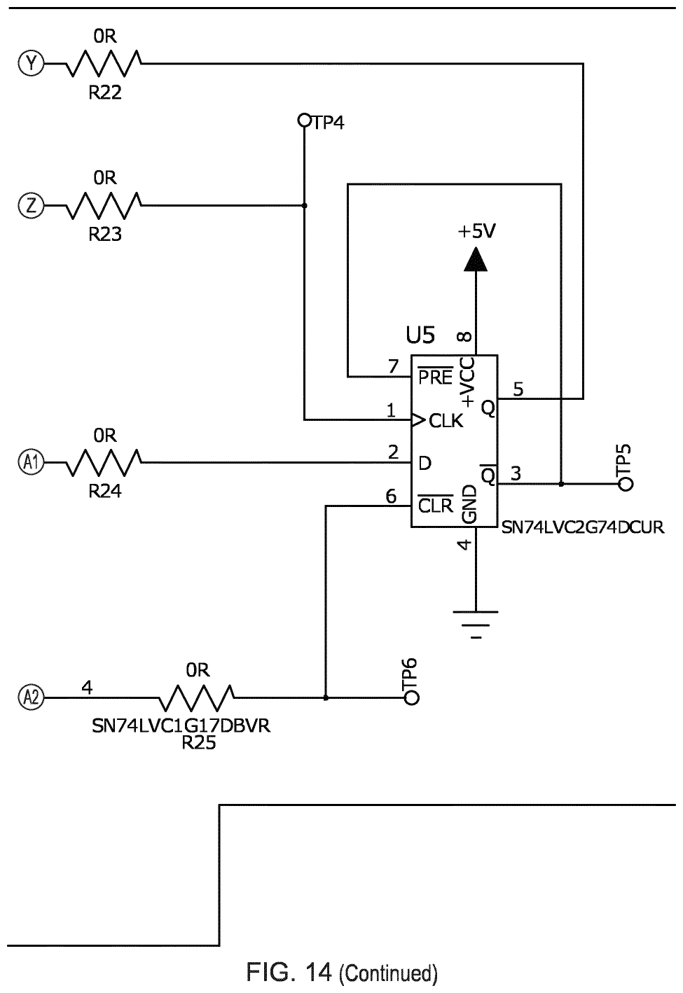

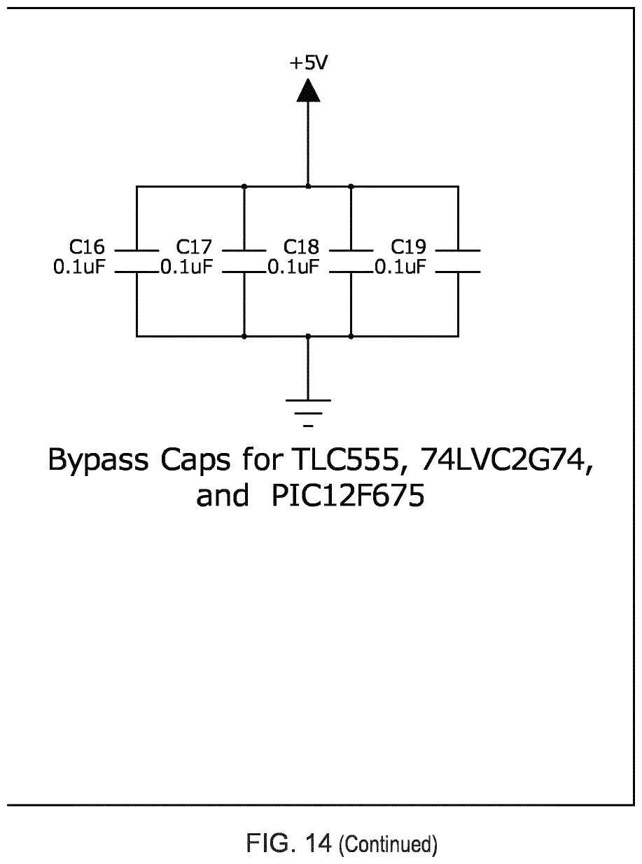

One example of the electrical signal generator 10 is shown in FIG. 6, while another example of the electrical signal generator 10 is shown in FIG. 14. The electrical signal generator 10 of FIG. 6 includes the power supply 12, electrical lead 14, container 28, and electrolytic solution 30. Some embodiments include two or more electrical signal generators 10, coupled to one or more leads 14, electrodes 18, and containers 28.

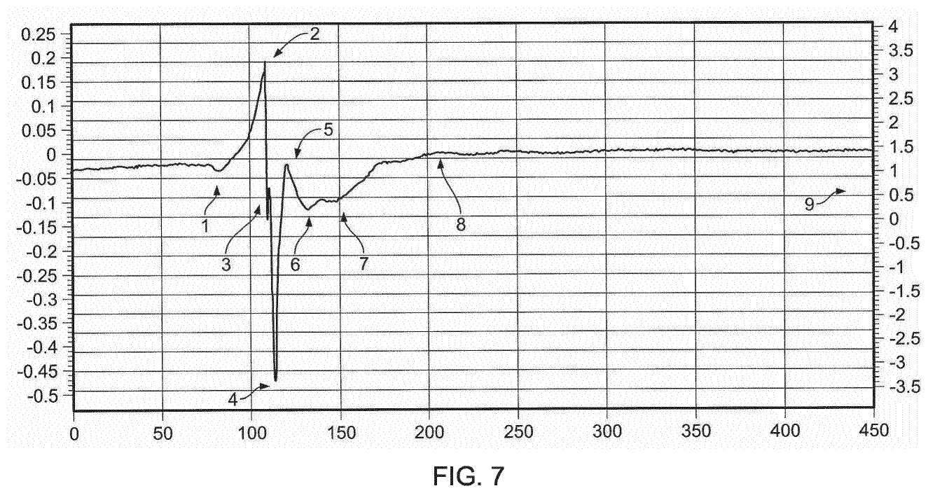

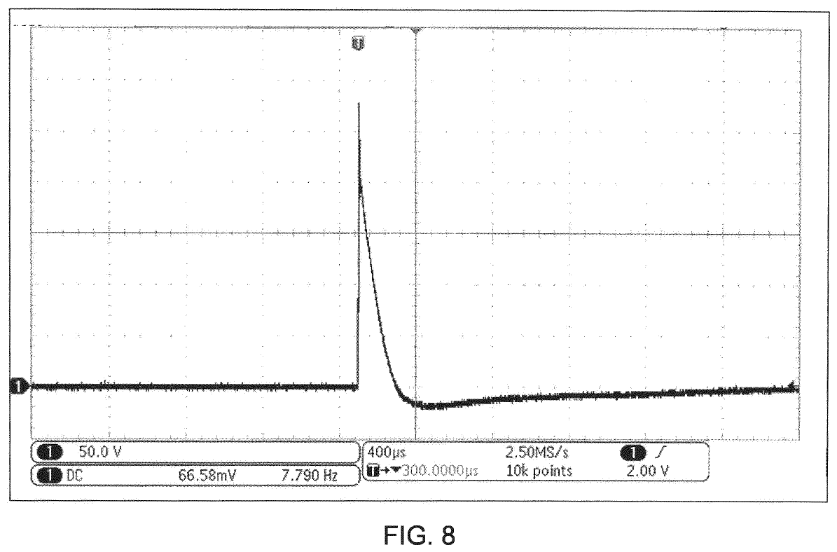

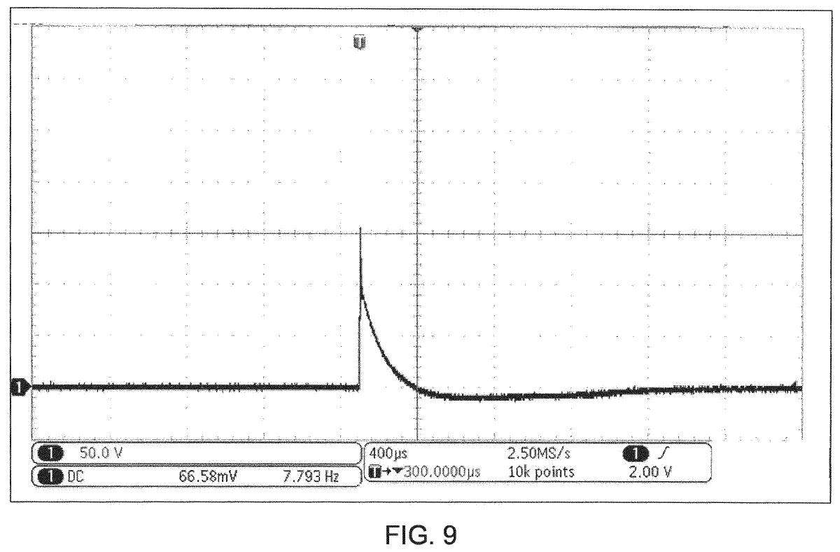

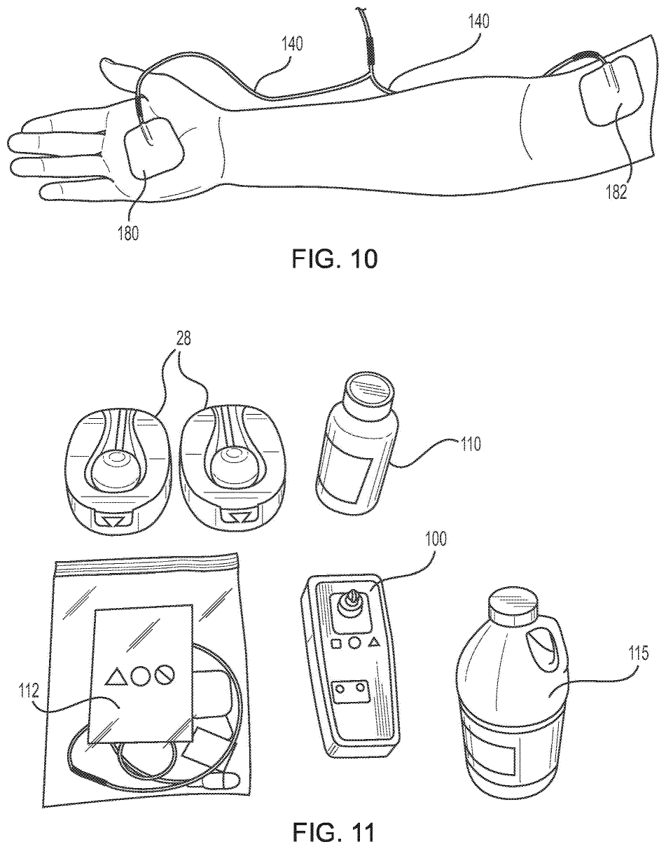

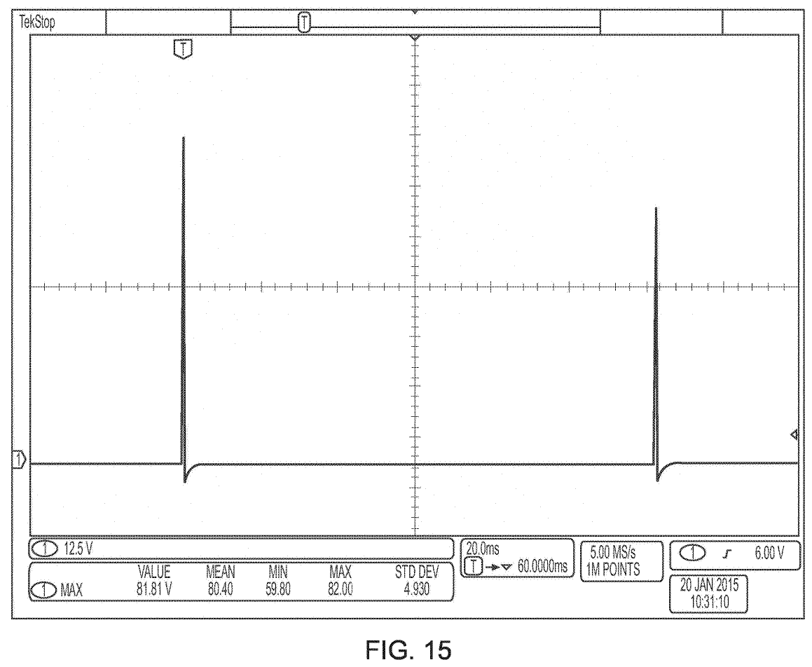

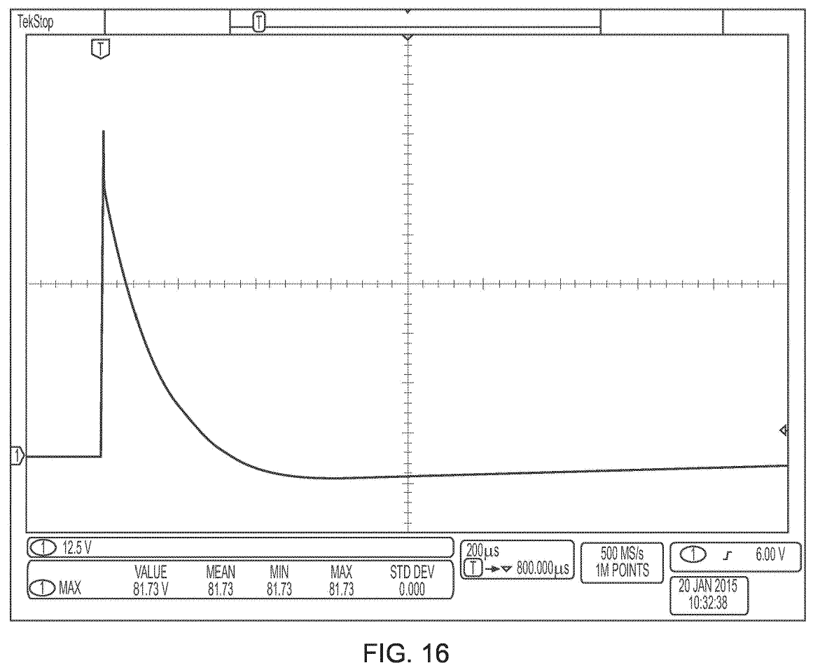

The electrical signal generator 10 comprises electrical circuitry 20 operable to generate an electrical output signal, such as having a waveform illustrated and described with reference to FIG. 7, or another suitable waveform, such as the waveforms shown in FIGS. 8, 9, 13, 15, and 16. In some embodiments the electrical circuitry 20 includes electronics such as one or more of resistors, capacitors, transformers, and a microprocessor in electrical communication with each other. In the example shown in FIG. 6, the electrical circuitry 20 of the electrical signal generator 10 includes a power switch 50, oscillator 52, variable control 54, and output circuitry 56. In this example the oscillator 52 includes an integrated circuit, such as a microcontroller 60. The output circuitry 56 includes a first stage 58, such as including operational amplifiers 64 and 66 and capacitor 68, and a second stage 60, including transformer 70. The output of the second stage 60 forms the output terminal 16, which can be electrically coupled to the lead 14 and electrode 18, to deliver the output signal to the patient.

The oscillator 52 operates to generate an initial oscillating signal. In this example, the oscillator includes a square wave generator. One example of a square wave generator is a microcontroller, such as the 8-pin, flash-based 8-bit CMOS microcontroller, part number PIC12F675, available from Microchip Technology Inc. of Chandler, Ariz., US. Another example of a square wave generator is a 555 timer. The square wave generator produces a squarewave signal, which oscillates between low and high voltages, such as between 0 and 5 volts. In this example the square wave has a frequency in a range from 4 Hz to 12 Hz. As one example the frequency is 7.83 Hz. Frequencies in this range have been found to be preferred over faster frequencies because they give the nerves in the patient time to repolarize after stimulation before the next stimulation. The frequency can be higher for a healthy person whose nerves can repolarize more quickly, while the frequency typically needs to be lower for an unhealthy person whose nerves require more time to repolarize.

In some embodiments the signal generator 10 includes a variable control 54, such as one or more potentiometers 22, 24 in electrical communication with the electrical circuitry of the signal generator 10. The one or more variable controls 54 allow an operator, such as a medical practitioner, the patient, or another person to provide an input to adjust the magnitude of the signal generated by the signal generator 10, such as to increase or decrease the magnitude of the signal. In this example, each potentiometer 22, 24 that is present in the signal generator corresponds to a separate output voltage channel (each having its own signal generator 10) having its own leads 14 and electrodes 18, and whose voltage is adjusted by its own intensity adjustment knob coupled to the variable control 54 that adjusts/sets the output voltage of that channel that is sent from the signal generator 10 to the patient via the leads 14 and electrodes 18. The ability to adjust the output voltage experienced by the patient may allow a patient to have the voltage adjusted down to a comfortable level, which may therefore contribute to lowering the patient's anxiety over use of the device, which may thus reduce the chance of any anxiety or stress induced vasoconstriction that can reduce the amount of blood within the targeted veins.

In one embodiment, the signal generator 10 includes two variable controls (e.g., potentiometers 22, 24), and therefore may have two separate output voltage channels each having its own signal generator 10, with each intensity knob and variable control 54 separately adjusting the output voltage to be sent to the patient along two sets of electrodes, corresponding to each of the two output voltage channels. A first of the two potentiometers 22 and its respective output voltage channel impart an output voltage to the patient that is configured to cause the target vein to become swollen or distended. A second of the two potentiometers 24 and its respective output voltage channel impart an output voltage to the patient that is configured to stop the pain at the needle stick site by interrupting nerve signals associated with pain. In the present embodiment, the two output voltage channels are identical, but in alternate embodiments, each potentiometer may be configured to adjust the output voltage in differing ranges. Having two separate channels, each with the ability to adjust the output voltage, allows the stimulation apparatus 1 to be configured to adapt to target veins in the foot, neck, elbow, or other such target vein sites.

In this example the electronic circuitry 20 of the signal generator 10 further includes output circuitry 56. The output circuitry operates to convert the square wave signal generated by the oscillator 52 into a desired output signal, such as having a waveform shown in FIG. 7-9, 13, 15, or 16.

The first stage 58 of the output circuitry includes electronics including operational amplifiers 64 and 66, and a capacitor 68. The first stage 58 is coupled to the variable control 54 to receive the input from a user to adjust the magnitude of the signal generated by the signal generator 10. In this example, the variable control 54 is a potentiometer that provides a variable resistance. The variable control 54 is electrically coupled to an input of the operational amplifier 64. The voltage of the signal provided by the variable control 54 changes as the variable control is adjusted. The operational amplifier 64 is configured as a unity gain buffer amplifier in this example.

The oscillator 52 generates a square wave output (e.g., pin 7) that is then supplied to the capacitor 68. The capacitor 68 converts the square wave signal to a series of pulses having a leading edge with a sharp voltage transition, followed by a trailing edge in which the voltage tapers off.

The signal is then provided to the second stage 66 where it is further filtered and amplified such as using the amplifier including operational amplifier 66 arranged in a non-inverting configuration.

The amplified signal is then provided to the second stage 60, including the transformer 70, which operates to amplify and rectify the signal.

In some embodiments the transformer 60 has an unequal ratio of windings. As one example, the transformer is a 10:1 transformer, which is arranged in a step-up configuration to increase the voltage at the output. In other possible embodiments the transformer can be arranged in a step-down configuration. Other embodiments have other ratios of windings. The output can also be generated in the second stage without using a transformer in yet other embodiments.

In this example, the transformer 60 is a center tap transformer. The oscillating signal generated by the first stage 58 is provided to the primary winding and the center tap, and operates in conjunction with a pair of diodes to rectify the output signal. The output signal is generated at the secondary windings and supplied to the output terminal 16. The ratio of the primary windings to the secondary windings determines the amplification provided by the transformer 70.

In some embodiments, the circuitry 20 further includes electronic components, and/or programming, that are configured to automatically vary the output signal, which may include varying one or more of the output voltage, the output current, shape of the output voltage waveform, and/or frequency of the output signal over time, without having to adjust the variable controls (e.g., potentiometers 22, 24). In one embodiment, the output signal may be changed over time by executing specific computer code or a software program in the microprocessor. In another embodiment, the output signal may be randomly changed inexpensively by the inclusion of a typical flashing light emitting diode (LED) 63 within the circuitry of the signal generator 10. Flashing LEDs automatically blink when supplied with electrical power, alternating between an “on” and “off” state, with the frequency of flashing between the two states depending on the input voltage. In one embodiment, the flashing LED is placed in the electrical circuit downstream of the microprocessor and upstream of the amplifying circuit that is connected to the output leads that are attached to the patient by the electrodes. The flashing LED, oscillating between an “on” and “off” state, is constantly switching the output current on and off, causing the signal generator 10 to vary the electrical output signal and voltage over time, according to the flashing frequency of the flashing LED. In this manner, the LED acts as a repetitive timer for the output signal from the signal generator. And because the frequency of the LED is dependent on its input voltage, adjusting the voltage from the potentiometer will change the frequency of the flashing LED, so as to provide an infinitely variable output signal to the patient.

Furthermore, the lower the quality of the components used to make the flashing LED, as with inexpensive flashing LEDs, the more variation or randomness there will be in the consistency or stableness of the frequency of the flashing for a given voltage. Accordingly, lower quality flashing LEDs provide a flashing pattern that is more random than that of higher quality flashing LEDs. Therefore, in one embodiment, to achieve more randomness in the frequency of the electrical signal sent to the patient from the signal generator 10, it may be beneficial to use lower quality flashing LED within the circuitry as disclosed herein.

In still alternate embodiments, additional methods to vary the output signal and voltage over time are contemplated herein, without departing from the scope of the present disclosure. By varying the output signal in the manner disclosed herein, the patient's body is constantly reacting to the changing output signal, rather than possibly becoming accustomed to a constant output signal to which the venous system might otherwise no longer respond after a short exposure thereto.

The signal generator 10 may also include at least one indicator 32, such as an LED or other lighted indicator, to indicate to the medical practitioner utilizing the electrical stimulation apparatus 1 as to when the power to the apparatus is turned “on.” An additional indicator may be included to indicate when the electrical signal is being sent to a patient. In one embodiment, the indicator may perform both functions, however, in alternate embodiments, separate indicators may be utilized to communicate each of the two functions.

The apparatus 1 may also include programming and/or a display screen configured to communicate and display for the medical practitioner the real time output voltage and signal, an initial set output voltage and signal, fault conditions, stimulation apparatus fault diagnostic information, or any other such setting, output, or feedback information as may be desired. In another embodiment, the apparatus 1 may include a display configured to graphically display the real time electrical information (e.g. the electrical signal and/or voltage vs. time) being sent to the patient. In still further embodiments, the stimulation apparatus 1 may include data output programming and associated output connectors that are configured to permit the apparatus to be connected to a separate, stand-alone external display for displaying any/all of the information disclosed herein.

In some embodiments the electronic circuitry 20 is arranged on one or more circuit boards. The circuit boards include at least one substrate layer, and typically have at least one layer of electrical traces formed thereon to make electrical connections between the electronic components. In some embodiments the electronic signal generator 10 is formed on the circuit board.

The output signal is sent from the signal generator 10 to the patient's body by two electrical leads 26 that are connected at a proximal end to the signal generator 10, and at a distal end to a pair of electrodes 18. In one embodiment of the present disclosure, the electrodes 18 may be configured as a pair of cups 28 or containers, such as for example, a pair of manicure nail soaking bowls or other such similar containers, that are configured to hold a liquid electrolyte solution 30 into which the finger and thumb tips of a patient are to be submerged. In some embodiments the containers include one or more recessed regions sized and shaped to receive at least the tips of the fingers of a hand, or the toes of a foot, therein. The purpose of using an electrolyte solution is to provide a conductive liquid medium into which the patient may place his fingers and through which the electrical signal may be delivered to the patient. In one embodiment, the electrolyte solution may be a mix of minerals and water. However, in alternate embodiments, the electrolyte solution may be any other type of solution used for increasing electrical conductivity between the electrical leads and the skin of a patient.

In another embodiment of the present disclosure, the electrodes may be configured as a pair of conductive electrode pads having a conductive gel or adhesive layer disposed on one side thereof to help adhere the electrode pad to the skin of a patient and to aid in making good electrical contact between the conductive pad and the patient's skin. Such electrode pads may be similar to those used with transcutaneous electrical nerve stimulation (TENS) devices or portable defibrillators. In addition, the electrode pads may be disposable. In one example embodiment, as shown in FIG. 10, at least one pair of electrodes 180,182 are configured as conductive electrode pads with an adhesive backing on one side thereof, such that a first electrode 180 of the pair of electrodes is attached to a palmar surface of one hand of a patient and a second electrode 182 of the pair of electrodes is attached to an arm, preferably to the bicep, of the patient. In the embodiment of FIG. 10, the arm to which the second electrode 182 is attached is the same arm as the hand to which the first electrode 180 is attached. Alternatively, the second electrode 182 may be attached to the patient's other arm. However, in this case, a greater level of intensity of the output signal would likely need to be supplied to the patient to achieve an effective vein distension. As also shown in FIG. 10, the pair of electrodes 180,182 are each connected to a distal end of an electrical lead 140, which are each connected at a proximal end to the signal generator (not shown) of the stimulation apparatus. In one example embodiment, the electrode 180 attached to the palmar surface of one hand of the patient will supply a positive output signal to the patient, while the other electrode 182 attached to the arm of the patient will supply a negative output signal to the patient. Alternatively, the negative output signal can be supplied to electrode 180, while the positive output signal can be supplied to electrode 182.

After attachment of the pair of electrodes 180,182 to the patient, the signal generator may be turned on to supply the output signal to the patient and to begin the electrical stimulation. The intensity of the output signal can be increased if no physical response, e.g., muscle fasciculation and/or vein distension, is observed. Alternatively, if the patient is experiencing discomfort, the intensity of the output signal can be decreased to a level that is tolerable, but that still produces a physical response, as discussed above. As the output signal is sent from the signal generator to the patient's body by the two electrical leads 140 that are connected at a proximal end to the signal generator (not shown), and at a distal end to the pair of electrodes 180,182, distension of the veins in the patient's arm will begin and will generally last for at least about ten (10) minutes, and may even last for more than about fifteen (15) minutes. In one example embodiment, the electrical stimulation is continued for at least two (2) minutes, but for no more than ten (10) minutes. In particular, the electrical stimulation can be discontinued once the target vein is visible and/or palpable. Once the target vein has become distended, the signal generator may be turned off, and venipuncture or any other medical procedure requiring vein distension may be performed.

In one embodiment, as shown in FIG. 11, the electrical stimulation apparatus of the present disclosure may comprise a kit for use by a medical practitioner. The kit can include a signal generator 100 that is preferably battery operated, a pair of containers 28 for holding an electrolytic solution, a prefilled labeled bottle of Epsom salt 110, a bottle of deionized water 115, and a disposable electrode assembly 112 that includes a pair of electrodes and a pair of electrical leads for connecting the pair of electrodes to the signal generator 100. The kit can be used for electrical stimulation of a patient by either using the containers 28 to which the electrodes are attached and the electrolytic solution is added, as discussed in one of the embodiments above, or by directly connecting the electrodes to the patient, as discussed in another of the embodiments above.

In the case of electrical stimulation of a patient using the containers 28 to which the electrolytic solution is added, the electrolytic solution can be prepared by adding the supplied deionized water 115 to the prefilled bottle of Epsom salt 110. In one embodiment, the Epsom salt concentration is at least about 30 g/L. The electrodes are thereafter attached to the containers 28, and a patient may then place their hands into the containers 28, prior to the addition of the prepared electrolytic solution into the containers 28. The electrodes are then attached to the signal generator 100 via the supplied electrical leads, and the signal generator 100 can be turned on to supply the output signal to the pair of electrodes. One of the electrodes can be supplied a negative output signal, while the other electrode can be supplied a positive output signal. As discussed above, once the target vein has become distended, the signal generator may be turned off, and venipuncture or any other medical procedure requiring vein distension may be performed. Prior to performing venipuncture, however, it may be preferred to wash the patient's hands with water in order to remove the salt solution, which may affect the outcome of any blood chemistry analysis.

While the previous embodiments disclosed the electrodes configured as either small containers for permitting the fingertips to be placed into an electrolyte solution, or conductive electrode pads, the electrodes should not be limited to such embodiments and in alternate embodiments may have alternate configurations as desired. For example, in alternate embodiments, the electrodes may be alternate sized containers that permit the submersion of a patient's full hands, feet, or any portion of the patient's body, including but not limited to arms and/or legs, into an electrolyte solution in electrical communication with the signal generator. In still alternate embodiments, the electrodes may be one or more of a metal pin-type probe or metal plate that are contact based electrodes. In still alternate embodiments, the electrode may be a finger clamp-type probe that is similar in mechanical structure to those used to measure pulse oximetry. In yet additional embodiments, the electrodes may be conductive garments, or other such contact-based electrode having an alternate physical configuration, without departing from the scope of the disclosure herein. In yet an additional embodiment, the electrodes may be configured as one or more electromagnets that generate a magnetic field, into which magnetic field the patient may place his hands, feet, or limbs. The electromagnetic field is configured to generate a complementary electric signal in the patient's body via changes to the magnetic field. In such an embodiment, the patient is not directly connected to the signal generator.

In one embodiment, the electrical signal output from the signal generator 10 sent to a patient's limbs through the electrodes includes an electrical signal that is an alternating signal (AC). In one embodiment, the AC signal sent to the patient has a frequency of 7.83 Hz (or 7.83 full alternating cycles per second). This means that the output circuit is interrupted 7.83 times per second. This frequency of 7.83 Hz has been selected in one embodiment to provide the nerves of the patient time to repolarize between successive output signals, and thus have time to get prepared for the next subsequent output signal. By providing adequate time to allow the nerves to repolarize, the signal generated by the signal generator 10 has a consistent effect on the skin, nerves, and muscles in the vicinity of the electrodes.

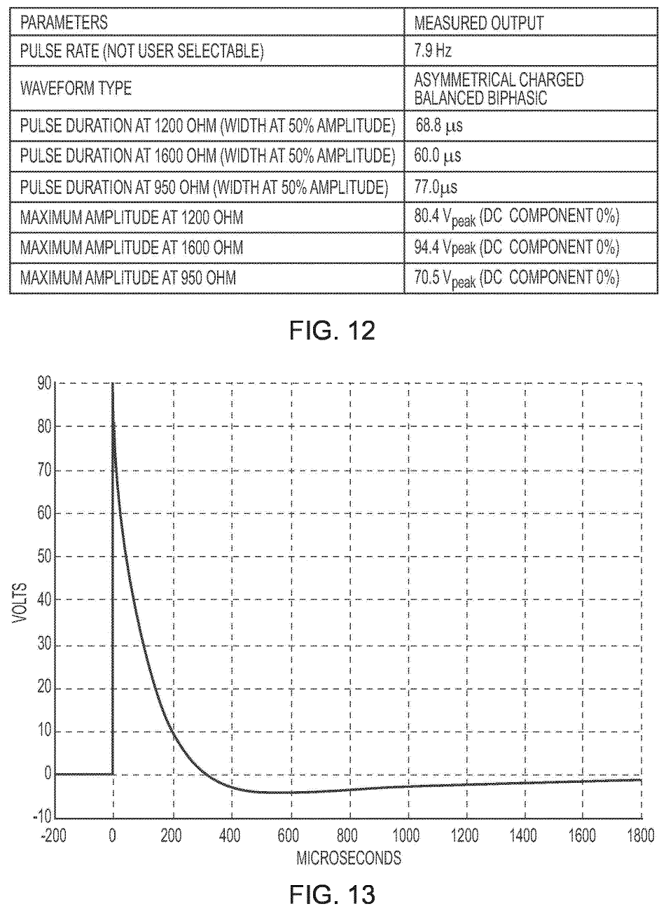

In another example embodiment, as shown in FIG. 12, the AC signal sent to the patient has a frequency of 7.9 Hz, with an asymmetrical charged balanced biphasic waveform. The duration of the pulse at 1200 ohms, 1600 ohms, and 950 ohms is 68.8 μs, 60.0 μs, and 77.0 μs, respectively. In addition, the maximum amplitude at 1200 ohms, 1600 ohms, and 950 ohms is 80.4 Vpeak, 94.4 Vpeak, and 70.5 Vpeak, respectively. While FIG. 12 displays one embodiment of theoretical standard measurements across purely resistive loads at maximum intensity settings, outputs may vary depending on parameter settings.

However, while the above embodiments operate at frequencies of 7.83 Hz and 7.9 Hz, respectively, the frequency of the output signal should not be read to be limited only to such specified frequencies, and in alternate embodiments, the AC or DC signal may have a different frequency without departing from the scope of the present disclosure. In alternate embodiments, the frequency of the output signal may be any alternate frequency, depending on the specific circuitry design of the signal generator. For example, in an alternate embodiment, a different duty cycle or output cycle, or even a different waveform that is subsequently developed, may use a different frequency. Furthermore, in alternate embodiments, the signal generator 10 may be configured to adjust the frequency or waveform of the output signal based on sensed feedback related to the physiological differences between patients of different ages, the patient's circulatory system patency, and other biomedical and/or bioelectrical aspects of the patient's body. In one embodiment, the microprocessor in the signal generator 10 may further contain programming that adjusts the output signal for the changes that are usually associated with an aging patient, such as thinner skin, more sensitive skin, skin that is sensitive to bleeding, etc.

In one embodiment, the output voltage from the signal generator 10, which is set by at least one of the potentiometers 22, 24, is initially set to be within the range of between 0 volts and 90 volts. In another embodiment, each of the two output voltage channels may be set to be within the range of between 0 volts and 90 volts. However, in alternate embodiments, the potentiometers 22, 24 may have larger or smaller output voltage ranges than that disclosed herein, and may each be selectably set to an initial output voltage value, or adjusted to a new output voltage value, within such larger or smaller voltage ranges, without departing from the scope of the present disclosure.

Feedback System

The signal generator 10 may further include an integrated feedback system that is configured to measure the resistance and capacitance of the patient's body during the time between each successive cycle of the output signal. In one embodiment, the feedback system utilizes a ten to one (10:1) audio transformer that responds to the electrical and capacitive resistance (i.e., electrical back pressure) of the patient's body, as well as any changes thereto, in order to adjust the output signal sent to the patient. Each human body presents with an electrical resistance. This resistance can change with the body's weight, hydration, etc. This electrical resistance can also change during the treatment. The signal generator 10 uses the audio transformer to measure the electrical resistance of the patient's body and, in response, appropriately alter the output voltage and/or current transmitted to the patient as part of the signal. In doing so, the signal may be altered based on the feedback from the feedback system to ensure that the signal generator 10 is eliciting the same clinical or physiological response in the patient's body, even when the patient's bodily response to treatment is changing (i.e. changes to the patient's electrical back pressure, or bodily resistance and/or capacitance).

A simple transformer performs the job of monitoring the electrical back pressure of the patient's body simply and inexpensively. When the microprocessor, via the transformer in electrical communication with the patient, detects a very high electrical resistance in the patient's body, then very little current will flow from the signal generator into the patient for a given constant output voltage from the signal generator to the patient. If the input current from the signal generator is very low (as when powered by a small battery), and if the output voltage leads do not have much resistance, then the battery power decreases and the current drops significantly. The measured electrical resistance of the human body is fairly constant, but the capacitance of the human body can vary greatly. This is a concern, because the sudden release of electrical energy or charge from the capacitor-like parts of the human body can result in the body receiving a painful jolt of electricity that may potentially cause damage to the patient's nervous or cardiac system, and otherwise interrupt the desired clinical response in the patient's body caused by the treatment.

The transformer of the feedback system filters an output voltage of the signal generator, which voltage fluctuates over time according to a preprogrammed voltage waveform, to allow the specific portions of the voltage waveform that are the most effective at eliciting the desired vein distension response to pass through to the patient. The electrical back pressure in the patient causes a reaction in the patient's body that creates a resulting electrical signal from the patient's body that can be captured and read by the signal generator, which can then be used as an input to adjust the output voltage of the next cycle of the output signal from the signal generator.

In alternate embodiments, the feedback mechanism may be specific programming within the microprocessor of the signal generator that is configured to monitor the feedback of the patient's electrical resistance and capacitance and, in turn, adjust the output signal sent to the patient based on the monitored feedback. In still alternate embodiments, the feedback system may utilize a plurality of sensors configured to measure the patient's resistance and capacitance, or any other such electrical component or computer code configured to measure feedback resistance and capacitance, without departing from the scope of the present disclosure.

In one embodiment, the apparatus 1 can be configured to stop all output signals from the signal generator 10 and wait for the patient's body to react to the last output signal. When the patient's body reacts to the last signal, the patient's body produces a resulting electrical signal that can be captured by the signal generator 10, analyzed, and used to alter the next output signal from the signal generator 10 that is sent to the patient. This can be done in real time with the appropriate microprocessor and software. In an alternate embodiment, if the feedback mechanism of the signal generator measures a change in a patient's bioelectrical resistance or capacitance of more than 10% between successive cycles of the output signals, the signal generator is configured to shut off or go into a fault mode, as a change of larger than 10% may indicate that the patient's body is experiencing a stress response and no is longer responding to the output signals. In one embodiment, the signal generator would automatically adjust the output signal waveform, voltage, and current based on the individual patient's specific physiology and related bioelectrical properties.

In still further embodiments, the signal generator includes software to collect physiological data from the patient using the stimulation apparatus, including the patient's physiological response data. That data can then be stored and analyzed by the signal generator and used to change the output signal in real time, so as to optimize the output signal and the achieved venous response for the specific patient.

Included in the signal generator may be a microprocessor having programming therein configured to control the amount of current and voltage being sent to the patient via the electrodes, as well as the shape of the output voltage waveform that is being sent to the patient, monitor the electrical feedback received from the patient (i.e. the patient's internal bodily resistance and/or capacitance), and/or adjust (e.g. automatically, in real time) any of the voltage output, the current output, and/or the shape of the voltage waveform being sent to the patient. The microprocessor may be any programmable microprocessor having any speed or internal memory size without departing form the scope of the present disclosure. In one embodiment, the microprocessor may include a comparator circuit configured to compare the original output signal sent to the patient from the signal generator to the returned signal from the patient. The results of the comparison are then used by the microprocessor to change the output signal proportionately to balance the next output signal sent to the patient. In such an embodiment, the microprocessor may have a baseline waveform stored in its memory which is sent to the patient with the first signal. A response/reflex signal is then sent back to the microprocessor from the patient through the feedback system, which response/reflex signal is also stored in the microprocessor. Thereafter, the microprocessor adapts the next outgoing signal based on the prior stored incoming response/reflex signal to gently coax the patient's nerves to carry the best waveform, voltage, and current necessary to produce the greatest visible presentation of the vein. This comparative process ensures that the output signal being set to the patient each time will continue to elicit the desired physiological and clinical response in the peripheral veins of the patient, preventing the patient's body from getting accustomed to the signal being sent.

Furthermore, the processor includes programming configured to maintain a predefined signal frequency. For example, in one embodiment, the microprocessor is programmed to maintain a preprogrammed signal frequency of 7.83 Hz. However, in alternate embodiments, alternate frequencies may be chosen without departing from the present disclosure. For example, in some patient groups or subsets, such as obese patients, geriatric patients, or neonatal patients, alternate signal frequencies may be needed to aid in eliciting the optimal venous presentation results. In addition, in an embodiment, the microprocessor may be programmed and configured to continue to operate properly on a constantly declining voltage, such as for example when the power supply is a battery that slowly runs out of power over time and continued use.

Waveform Graph

FIG. 7 shows an exemplary graph of an embodiments of active portions of a single cycle of a signal. The graph shows an output voltage (the Y-axis) of the output signal, versus time in milliseconds (the X-Axis), that may be able to illicit vein distension. The shape of the signal, including the location and amplitude of the various peaks and valleys therein, is an exemplary waveform that may elicit active, signal-based enlargement of the target peripheral veins. FIGS. 8, 9, 13, 15, and 16 show additional exemplary waveform graphs of active portions of a single cycle of a signal.

Referring further to FIG. 7, a plurality of points 1-9 are identified on the graphed waveform showing the output signal's output voltage vs. time. Point 1 on the graph corresponds to the beginning of a new cycle of the repetitive output signal, and indicates the initial output voltage from the signal generator that is selected to alert or stimulate a patient's sensory nerve (via its dendrites in the surface of the skin) to a change in condition. This initial output voltage initiates a tiny electrical signal in the patient's body, having a unique voltage, current, and waveform, to be sent to the central nervous system so the brain can monitor the extremities. In response, the brain sends a healing signal back to that specific sensory dendrite from which the signal to the brain originated.

Point 2 on the graph corresponds to the primary effective portion of the nerve stimulation signal. This point is the main output voltage in the nerve stimulating portion of the output signal that causes the peripheral nerves in the patient's limbs to over-react and causes a simultaneous tetany or spasm of the nearby muscles surrounding the target peripheral veins. This is the portion of the waveform that is adjusted via the knob of one of the potentiometers 22, 24 on the signal generator. In overweight patients, the voltage level at Point 2 is automatically suppressed by a layer of fat in the skin. Accordingly, for overweight patients, in order to get the signal to reach the nerves of the patient and overcome the resistance of the fat layer, it may be necessary to send a higher output voltage to the patient. This can be accomplished by using a ten to one (10:1) audio transformer, or other such transformer, in the signal generator to amplify the output voltage signal sent to the patient. Alternatively, the increasing of the voltage to overcome the resistance of the fat layer so the signal may reach the nerves may also be accomplished by the implementation of programming contained in the microprocessor.

Point 3 in the voltage waveform graph corresponds to the output voltage that triggers the sensory nerve in the patient to “turn off” In this regard, Point 3 is the voltage that triggers the nerve to be at rest and reset to its standby voltage, waiting to be used or triggered “on” again in the next subsequent cycle of the output signal. Point 4 in the voltage waveform graph is the output voltage that cancels the positive portion of the signal and balances the stimulation apparatus' nerve signal to allow the nerve time to reset itself, or repolarize.

Point 5 in the waveform graph corresponds to the muscle stimulation portion of the output signal, and is the output voltage that causes the motor muscles to stimulate the venous muscle pump that may, in turn, cause the veins to distend and fill with blood. In the waveform presented in FIG. 7, the length of time during which this portion of the signal is active is small, however in some patients the length of time over which this portion of the output voltage in the output signal is active will be adjusted to achieve the proper amount of voluntary muscle stimulation to activate the venous muscle pump. The longer that this portion of the signal is active, the more that the muscles are stimulated. In addition, the small involuntary smooth muscles surrounding the veins require a different amount of active stimulation time to activate the venous muscle pump action than that of the larger muscles. This portion of the waveform also may be adjusted from patient to patient to achieve the optimal venous muscle pump action in each patient.

Point 6 in the waveform graph is the point at which the motor muscle stimulation is shut off to allow them to reset and get ready for the next cycle of the signal. Point 7 in the waveform graph corresponds to a reflex signal back pressure from the patient's peripheral nervous system, indicating that the nervous system is trying to take over control of the nerves and muscles and stabilize the patient's muscle and nerve activity. Point 8 in the waveform graph corresponds to a period of zero output voltage to the patient, and is part of the integrated feedback loop that the peripheral nervous system uses to gently restore the patient's baseline electrical potential back to its original resting electrical potential, or internal voltage. In comfortable, relaxed patients, their resting potential, or measured voltage, may be on the order of 20 millivolts. However, in some patients who are anxious, their measured resting potential may be zero volts, or a positive measured voltage, which are otherwise higher electrical potentials or voltages than a typical relaxed patient. This initial resting potential measurement may be used to setup the basic parameters of the first and each succeeding treatment output signal from the signal generator.

Point 9 in the waveform graph corresponds to the patient's baseline condition, whereby there is no active output signal or voltage being sent to the patient's body, and the patient is otherwise unaffected by any output signal from the stimulation apparatus. This also corresponds to the period during which the signal generator is monitoring the patient's internal electrical potential and preparing to initiate a new cycle of the signal, and adjusting the active portion of the output signal based on the feedback monitored from the patient.

In some embodiments, the waveform has one or more of the following properties. The highest voltage reached stimulates the muscles surrounding the veins. The width of the signal from the baseline until the return the baseline stimulates the nearby voluntary motor muscles to function as a venous muscle pump to empty the adjacent veins of blood. The return to baseline stops the action of both muscles. The negative pulse following the first return to baseline begins the return to the original resting state of the muscles and nerves. The negative pulse delivers a negative polarity pulse that with a volume of energy (e.g., watts) that equals the energy delivered in the original positive polarity phase. The second return to baseline finishes the polarity balancing. The time period until the next signal allows the nerve and muscle cells to re-organize and prepare for the next sequence of stimulation. Other waveforms have other properties.

Apparatus Operation and Stimulation Action

In operation, the stimulation apparatus functions as follows. The electrodes are placed in electrical contact a subject, e.g. with the fingers, hands, and/or limbs of a patient. In one embodiment, this involves the patient placing the fingertips of each hand into separate containers of an electrolyte solution. The electrolyte solution in each container is placed in electrical communication with the signal generator by separate electrical leads that are terminated at one end in the electrolyte solution, and at the opposite end to output contacts of the signal generator. In alternate embodiments, the electrodes may be adhesive backed pads that are affixed directly to the patient's skin.

The power source supplies power to the signal generator. The medical practitioner adjusts the output voltage to the patient by rotating an adjustment knob of at least one potentiometer. The signal generator is switched “on” and the preprogrammed electrical output signal is transmitted through the leads and electrodes to the fingertips, hands, and/or arms of the patient. The preprogrammed output signal includes a repetitive cycle of preprogrammed fluctuating output voltages at various specified points in time for each cycle. In one embodiment, the initial output voltage may be set between 0 and 90 volts and the signal delivered is less than one milliamp. However, in alternate embodiments, the output voltage range may be larger or smaller, or cover a different voltage range than that disclosed in the present embodiment, and the output signal may be larger than 1 milliamp without departing from the scope of the present disclosure.

Each cycle of the output electrical signal includes a period of active output voltage and a period of rest, where no output voltage is being imparted to the patient's limbs. The preprogrammed output voltage may include several phases including, one or more of the following: an initiation phase that alerts the patient's sensory nerve to the presence of the output voltage; a primary nerve stimulation phase that causes the peripheral nerves to force the motor muscles surrounding the peripheral target veins to contract; an end to the nerve stimulation phase that turns “off” the sensory nerve; a balancing phase that cancels the stimulation signals that were sent to the nerves to allow the nerves to reset; a muscle stimulation phase that activates the venous muscle pump; a shutdown phase that ends the activation of the motor muscles; an electrical back pressure phase; an electrical feedback phase; and a rest phase with no active voltage output to allow the patient's system time to reset before the next cycle begins. This cycling part of the waveform in the current embodiment is not exclusive of other possible waveforms. What is envisioned is a waveform that may cause all the actions described in this application and may vary relative to the patient's physiology, the design and limitations of the electronic circuitry, and/or the method used to deliver the signals to the patient.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A method of forming an arteriovenous connection in a subject, comprising:
electrically stimulating vessels including at least one artery of the subject and at least one vein of the subject; and
subsequently fluidly connecting the at least one artery to the at least one vein.

2. The method of claim 1, wherein fluidly connecting the at least one artery to the at least one vein comprises directly connecting the at least one artery to the at least one vein to form an arteriovenous fistula (AVF).

3. The method of claim 2, wherein the AVF is a brachiocephalic fistula or a radiocephalic fistula.

4. (canceled)

5. The method of claim 1, wherein fluidly connecting the at least one artery to the at least one vein comprises connecting the at least one artery to the at least one vein with an intermediate conduit to form an arteriovenous graft.

6. The method of claim 1, wherein the vessels are electrically stimulated for at least 30 seconds.

7. (canceled)

8. The method of claim 1, further comprising applying a constriction to a limb of the subject that includes the at least one artery and the at least one vein, wherein the constriction is applied after electrically stimulating the vessels and before fluidly connecting the at least one artery and the at least one vein.

9. The method of claim 8, wherein the constriction is applied for at least 1 minute.

10. (canceled)

11. The method of claim 8, wherein applying the constriction comprises applying a tourniquet.

12. The method of claim 1, wherein electrically stimulating vessels includes generating an adjustable electrical output signal with an electrical stimulation apparatus, the signal including an adjustable output voltage, an adjustable current, and an adjustable output voltage waveform configured to elicit a physiological response in the subject.

13. The method of claim 12, wherein the electrical stimulation apparatus comprises:
(i) a powered signal generator configured to generate the adjustable electrical output signal; and
(ii) at least two electrodes in electrical communication with the signal generator and configured to be placed in electrical communication with the subject; and wherein electrically stimulating vessels includes:
electrically connecting the at least two electrodes to the subject; and
transmitting the output signal to the subject via the at least two electrodes to elicit the physiological response.

14. The method of claim 13, further comprising:
monitoring biological electrical feedback from the subject in the form of the subject's biological electrical resistance and/or capacitance;
comparing the electrical feedback from the subject with the transmitted output signal;
adjusting subsequent output signals to be sent to the subject based on the comparison between the transmitted output signal and the electrical feedback; and
transmitting said subsequent output signals to the subject via the at least two electrodes.

15. The method of claim 14, wherein the step of monitoring the biological electrical feedback from the subject is performed by an electrical feedback system that is optionally in the powered signal generator.

16. The method of claim 15, wherein comparing the electrical feedback from the subject with the transmitted output signal is performed using a microprocessor that is optionally integrated in the powered signal generator.

17. The method of any of claim 16, wherein electrically connecting the at least two electrodes to the subject comprises connecting a first electrode to the hand of the subject and connecting a second electrode to the arm of the subject.

18. The method of claim 17, wherein connecting the first electrode to the hand of the subject comprises connecting the first electrode to a palmar surface of the hand of the subject.

19. The method of claim 1, wherein electrically stimulating vessels comprises providing a variable output voltage to the subject between 0 and 90 volts.

20. The method of claim 1, wherein electrically stimulating vessels comprises providing a variable current to the subject that is less than 1 milliamp.

21. (canceled)

22. The method of claim 1, further comprising:
accessing the at least one vein.

23. The method of claim 22, wherein accessing the at least one vein comprises cannulating the at least one vein.

24. The method of claim 22, wherein fluidly connecting the at least one artery to the at least one vein comprises connecting the at least one artery to the at least one vein with an intermediate conduit to form an arteriovenous graft, and wherein accessing the at least one vein comprises cannulating the intermediate conduit.

25. (canceled)

26. The method of claim 22, wherein accessing the at least one vein comprises forming a first access port and a second access port in the vein.

27. The method of claim 26, further comprising removing a fluid from the at least one vein through the first access port and introducing the fluid into the at least one vein through the second access port.

28. (canceled)

29. The method of claim 27, wherein the fluid is blood, the method further comprising performing hemodialysis on the blood after removal of the blood from the at least one vein and before introduction of the blood into the at least one vein.

30. The method of claim 29, wherein accessing the at least one vein is performed after a maturation period following the step of forming the arteriovenous connection, wherein the maturation period is at least 2 weeks.

31. (canceled)

32. (canceled)
